(19) United States
(12) Patent Application Publication
(10) Pub. No.: US 2025/0285274 A1
(43) Pub. Date: Sep. 11, 2025

(54) IMAGE PROCESSING APPARATUS, METHOD, AND PROGRAM

(71) Applicant: FUJIFILM Corporation, Tokyo (JP)

(72) Inventor: Jun YAMASHITA, Tokyo (JP)

(73) Assignee: FUJIFILM Corporation, Tokyo (JP)

(21) Appl. No.: 19/074,428

(22) Filed: Mar. 9, 2025

(30) Foreign Application Priority Data
Mar. 11, 2024 (JP) 2024-037208

(51) Int. Cl. G06T 7/00; G06T 7/11; G06T 7/55; G06T 11/00; G06T 19/00

(52) U.S. Cl. CPC G06T 7/0012; G06T 7/11; G06T 7/55; G06T 11/008; G06T 19/00; G06T 2207/10081; G06T 2207/30084; G06T 2207/30196

(57) ABSTRACT

There are provided image processing apparatus, method, and program which can analyze a tissue of interest for each component. An image processing apparatus configured to process an image obtained by an X-ray computed tomography device capable of measuring an effective atomic number includes a processor. The processor displays at least one of a plurality of images obtained by the X-ray computed tomography device on a display unit, receives setting of a first region on the image displayed on the display unit, receives setting of an effective atomic number as an analysis target, extracts a pixel including a component of the effective atomic number in the first region and extracts a second region including the component of the effective atomic number in the first region, specifies a shape of the second region, and displays the shape of the second region on the display unit.

### CROSS-REFERENCE TO RELATED APPLICATION

The present application claims priority under 35 U.S.C § 119(a) to Japanese Patent Application No. 2024-037208 filed on Mar. 11, 2024, which is hereby expressly incorporated by reference, in its entirety, into the present application.

### BACKGROUND OF THE INVENTION

### 1. Field of the Invention

The present invention relates to image processing apparatus, method, and program, and particularly to image processing apparatus, method, and program that process an image obtained by an X-ray computed tomography device (X-ray CT device) capable of measuring an effective atomic number.

### 2. Description of the Related Art

JP2012-147930A discloses a technique of specifying a lung nodule candidate from an image acquired by an X-ray CT device and obtaining a shape and the like of the lung nodule candidate.

### SUMMARY OF THE INVENTION

In a disease that causes a stone, such as a ureteral stone, a treatment method, a prevention method, and the like for the disease vary depending on the component of the stone. However, in the technique disclosed in JP2012-147930A, it is not possible to analyze the shape and the like of a tissue of interest for each component.

An embodiment according to the disclosed technology provides image processing apparatus, method, and program which can analyze a tissue of interest for each component.

(1) An image processing apparatus that processes an image obtained by an X-ray computed tomography device capable of measuring an effective atomic number, the image processing apparatus including a processor, in which the processor displays at least one of a plurality of images obtained by the X-ray computed tomography device on a display unit, receives setting of a first region on the image displayed on the display unit, receives setting of an effective atomic number as an analysis target, extracts a pixel including a component of the effective atomic number in the first region and extracts a second region including the component of the effective atomic number in the first region, specifies a shape of the second region, and displays the shape of the second region on the display unit.

(2) The image processing apparatus according to (1), in which the processor measures a size of the second region, and displays the shape and the size of the second region on the display unit.

(3) The image processing apparatus according to (1) or (2), in which the processor receives setting of a range of the effective atomic number as the analysis target.

(4) The image processing apparatus according to (3), in which the processor receives setting of a value of the effective atomic number as a reference and a range based on the value, and receives the setting of the range of the effective atomic number as the analysis target.

(5) The image processing apparatus according to any one of (1) to (4), in which the processor displays, as the effective atomic number corresponding to a specific component, the effective atomic number registered in advance on the display unit, and receives selection from the effective atomic number displayed on the display unit and receives the setting of the effective atomic number as the analysis target.

(6) The image processing apparatus according to (5), in which the processor displays information on the component corresponding to the effective atomic number on the display unit in association with the effective atomic number.

(7) The image processing apparatus according to (5) or (6), in which the processor receives registration of the effective atomic number to be displayed on the display unit.

(8) The image processing apparatus according to any one of (1) to (7), in which the processor receives setting of a plurality of first regions, receives setting of the effective atomic number as the analysis target for each first region, extracts the second region for each first region, specifies the shape of the second region for each first region, and displays the shape of the second region on the display unit for each first region.

(9) The image processing apparatus according to (8), in which the processor measures a size of the second region for each first region, and displays the shape and the size of the second region on the display unit for each first region.

(10) The image processing apparatus according to any one of (1) to (9), in which the processor receives setting of a range of the image as the analysis target from among the plurality of images obtained by the X-ray computed tomography device, individually extracts the second region from the image in the set range, specifies a three-dimensional shape of the second region, and displays the three-dimensional shape of the second region on the display unit.

(11) The image processing apparatus according to (10), in which the processor measures a volume of the second region and measures a size of the second region, and displays the three-dimensional shape and the volume of the second region on the display unit.

(12) The image processing apparatus according to (10) or (11), in which the processor displays a three-dimensional image generated from the plurality of images obtained by the X-ray computed tomography device on the display unit, and receives setting of the first region on the three-dimensional image displayed on the display unit.

(13) The image processing apparatus according to any one of (1) to (12), in which the processor displays a three-dimensional shape of the second region on a three-dimensional image generated from the plurality of images obtained by the X-ray computed tomography device.

(14) The image processing apparatus according to any one of (1) to (13), in which the X-ray computed tomography device is an X-ray computed tomography device capable of photon counting computed tomography.

(15) The image processing apparatus according to any one of (1) to (14), in which the image obtained by the X-ray computed tomography device is an effective atomic number image.

(16) An image processing method of processing an image obtained by an X-ray computed tomography device capable of measuring an effective atomic number, the image processing method including: displaying at least one of a plurality of images obtained by the X-ray computed tomography device on a display unit; receiving setting of a first region on the image displayed on the display unit; receiving setting of an effective atomic number as an analysis target; extracting a pixel including a component of the effective atomic number in the first region and extracting a second region including the component of the effective atomic number in the first region; specifying a shape of the second region; and displaying the shape of the second region on the display unit.

(17) An image processing program for processing an image obtained by an X-ray computed tomography device capable of measuring an effective atomic number, the image processing program causing a computer to execute: a function of displaying at least one of a plurality of images obtained by the X-ray computed tomography device on a display unit; a function of receiving setting of a first region on the image displayed on the display unit; a function of receiving setting of an effective atomic number as an analysis target; a function of extracting a pixel including a component of the effective atomic number in the first region and extracting a second region including the component of the effective atomic number in the first region; a function of specifying a shape of the second region; and a function of displaying the shape of the second region on the display unit.

According to the present invention, it is possible to analyze a tissue of interest for each component.

### BRIEF DESCRIPTION OF THE DRAWINGS

FIG. 1 is a schematic configuration diagram of a PCCT device.

FIG. 2 is a diagram illustrating an example of a hardware configuration of a console.

FIG. 3 is a block diagram of main functions of a console regarding generation of a tomographic image.

FIG. 4 is a conceptual diagram of an analysis function.

FIG. 5 is a block diagram of main functions of a console regarding an analysis function.

FIG. 6 is a diagram illustrating an example of a display screen of an effective atomic number image.

FIG. 7 is a diagram illustrating an example of a setting screen (analysis condition setting dialog) of an analysis condition.

FIG. 8 is a diagram illustrating an example of a setting screen of an analysis target region.

FIG. 9 is a diagram illustrating an example of display of a region setting frame.

FIG. 10 is a diagram illustrating an example of screen display of an analysis result.

FIG. 11 is a flowchart illustrating an operation procedure in a case of performing processing of analyzing a shape and a size of a specific tissue.

FIG. 12 is a diagram illustrating an example of screen display in a case of setting an analysis target region on a three-dimensional image.

FIG. 13 is a diagram illustrating an example of a setting screen (analysis condition setting dialog) of an analysis condition in a case of setting an effective atomic number using a preset.

FIG. 14 is a diagram illustrating an example of a screen (effective atomic number setting dialog) for setting an effective atomic number using a preset.

FIG. 15 is a diagram illustrating an example of a setting screen (analysis condition setting dialog D) for an analysis condition in a case of setting an effective atomic number having a width.

FIG. 16 is a diagram illustrating another example of a setting screen of an analysis condition in a case of setting an effective atomic number having a width.

FIG. 17 is a diagram illustrating an example of a case of outputting an analysis result in a superimposed manner on a three-dimensional image as an analysis source.

FIG. 18 is a diagram illustrating an example of a case of also displaying a measurement result of a size in a case where an analysis result is output in a superimposed manner on a three-dimensional image as an analysis source.

### DESCRIPTION OF THE PREFERRED EMBODIMENTS

Hereinafter, preferred embodiments for carrying out the present invention will be described with reference to the accompanying drawings.

### PCCT Device

Here, a case where the present invention is applied to an X-ray computed tomography device (PCCT device) capable of performing photon counting computed tomography (PCCT) will be described as an example.

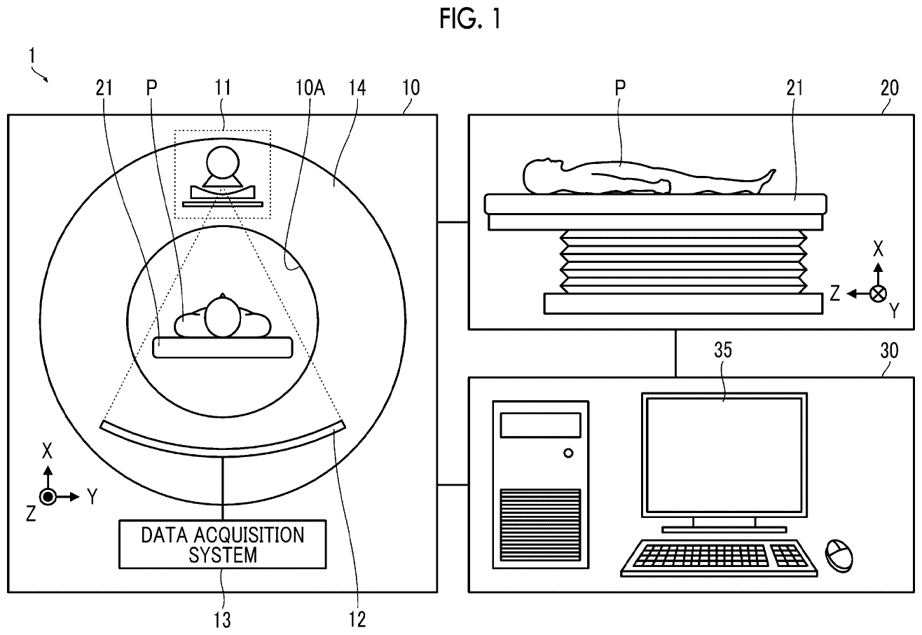

FIG. 1 is a schematic configuration diagram of a PCCT device. Note that in FIG. 1, an X axis, a Y axis, and a Z axis are three axes orthogonal to each other. A Y-axis direction and a Z-axis direction are defined as a horizontal direction, and an X-axis direction is defined as a vertical direction (up-down direction). In addition, the Z-axis direction is defined as a body axis direction.

As illustrated in FIG. 1, a PCCT device 1 includes a scanner gantry 10, an examination table 20, a console 30, and the like. The respective apparatuses are connected to each other in a communicable manner.

### Scanner Gantry

The scanner gantry 10 has an opening portion (bore), and irradiates a subject P inserted into an opening portion 10A with X-rays to execute the scan of the PCCT. The scanner gantry 10 includes an X-ray tube device 11, an X-ray detection device 12, a data acquisition system (DAS) 13, a rotation frame 14, and the like.

The X-ray tube device 11 irradiates the subject P with X-rays. The X-ray tube device 11 includes an X-ray tube, an X-ray high-voltage device, a bowtie filter, a collimator, and the like. The X-ray tube, which is an X-ray source, outputs X-rays by applying a high voltage from the X-ray high-voltage device. The subject P is irradiated with the X-rays output from the X-ray tube through the bowtie filter and the collimator.

The X-ray detection device 12 detects the X-rays emitted from the X-ray tube device 11 and transmitted through the subject P. The X-ray detection device 12 is a photon counting type X-ray detection device. The photon counting type X-ray detection device outputs an electric signal corresponding to the number of photons as a detection signal of X-rays. The X-ray detection device 12 has, for example, a structure in which a plurality of detection elements are two-dimensionally arranged in a channel direction (circumferential direction) and a column direction (body axis direction).

The data acquisition system 13 collects the electric signals output from each detection element of the X-ray detection device 12, and generates detection data. The detection data is data in which a count value (count number) of X-ray photons is assigned for each energy bin. The energy bin is a section in which the X-ray spectrum is divided into energy bandwidths. The detection data generated by the data acquisition system 13 is output to the console 30.

The rotation frame 14 has a cylindrical shape and is rotated around an axis by being driven by a rotary drive unit (not illustrated). An inner peripheral portion of the rotation frame 14 constitutes the opening portion 10A of the scanner gantry 10. The X-ray tube device 11 and the X-ray detection device 12 are mounted on the rotation frame 14. The X-ray tube device 11 and the X-ray detection device 12 are disposed to face each other with the opening portion 10A interposed therebetween. By rotating the rotation frame 14, the X-ray tube device 11 and the X-ray detection device 12 are rotated around a rotation axis of the rotation frame 14. The rotation axis of the rotation frame 14 constitutes an imaging center.

### Examination Table

The subject P is placed on the examination table 20, and the examination table 20 is moved in both the up-down direction and the horizontal direction. The examination table 20 includes a top plate 21 on which the subject P is placed. The top plate 21 is driven by an up-down drive unit (not illustrated), and is moved up and down in the vertical direction. In addition, the top plate 21 is driven by a horizontal drive unit (not illustrated), and is moved horizontally in the body axis direction (Z-axis direction). The position (height) of the subject P in the up-down direction is adjusted by moving the top plate 21 up and down. In addition, the subject P is moved in the opening portion 10A of the scanner gantry 10 along the body axis direction by moving the top plate 21 horizontally along the body axis direction.

### Console

The console 30 functions as an operation panel, and also functions as an image processing apparatus that performs various kinds of image processing.

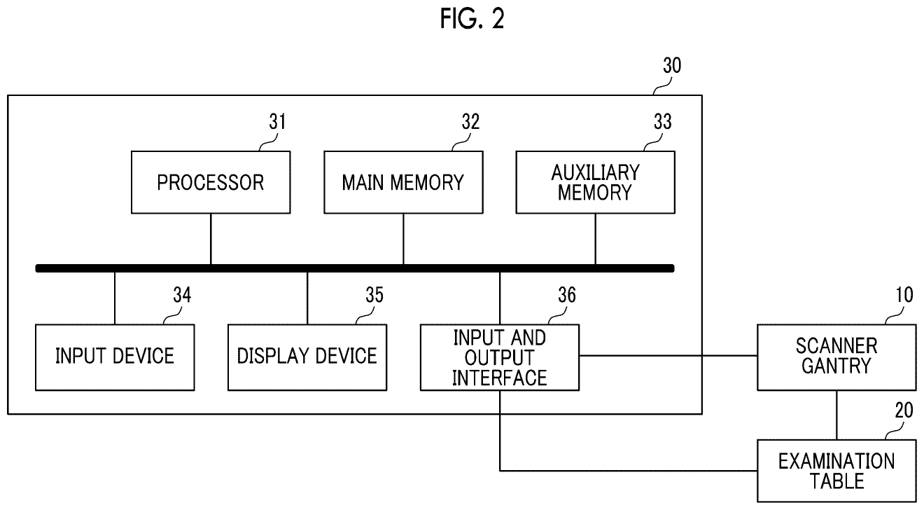

FIG. 2 is a diagram illustrating an example of a hardware configuration of the console.

The console 30 is configured by a computer, and includes a processor 31, a main memory 32, an auxiliary memory 33, an input device 34, a display device 35, an input and output interface 36, and the like.

As the processor 31, for example, a central processing unit (CPU) which is a general-purpose processor that executes a program to function as various processing units is adopted. Various programs executed by the processor 31 and data are stored in the main memory 32 and/or the auxiliary memory 33. The program is synonymous with software.

The main memory 32 includes a random access memory (RAM) and a read only memory (ROM).

The auxiliary memory 33 is configured of, for example, a hard disk drive (HDD) or a solid state drive (SSD).

The input device 34 includes, for example, a keyboard, a mouse, a touch panel, and the like.

The display device 35 is configured of, for example, a liquid crystal display (LCD) or an organic electro luminescence diode display (OLED display). In the present embodiment, the display device 35 is an example of a display unit.

The input and output interface 36 communicably connects the console 30 to the scanner gantry 10 and the examination table 20.

### Function as Operation Panel

The console 30 integrally controls the overall operation of the PCCT device 1 on the basis of an operation input from a user. Setting of imaging conditions, setting of conditions for reconstruction processing, setting of analysis conditions, and the like are performed via the console 30.

### Function as Image Processing Apparatus

### (a) Function of Generating Tomographic Image

From the detection data obtained by the PCCT, it is possible to acquire spectral images having various kinds of information, such as a virtual monochromatic X-ray image, an effective atomic number image, and a material discrimination image, in addition to a normal tomographic image (CT image) illustrating the distribution of the linear attenuation coefficients. The virtual monochromatic X-ray image is an image obtained by virtually expressing an image obtained at a single energy. The effective atomic number image is an image illustrating a distribution of an effective atomic number (effective Z) of a material. The effective atomic number is an atomic number corresponding to constituent elements of a compound or a mixture when viewed in an averaged manner. In the effective atomic number image, the effective atomic number is represented for each pixel. The material discrimination image is an image representing a distribution of density values of a material. In the material discrimination image, a density value of a material is represented for each pixel.

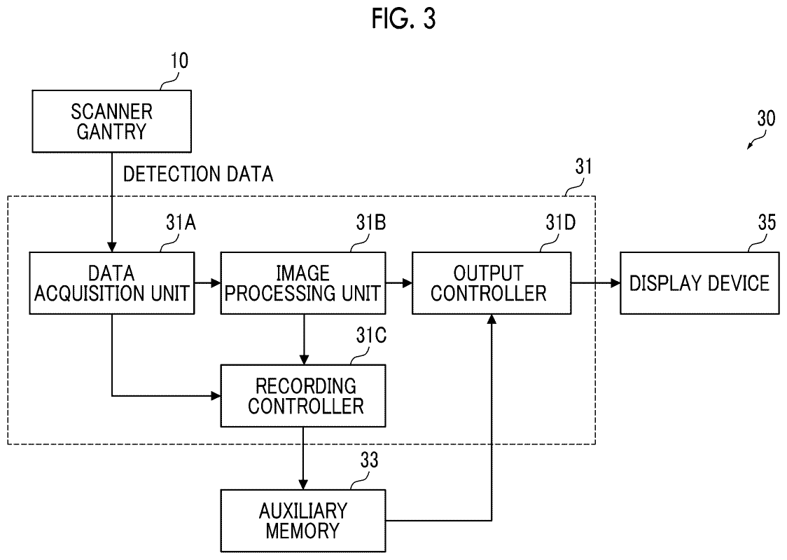

FIG. 3 is a block diagram of main functions of the console regarding generation of the tomographic image.

The console 30 has functions of a data acquisition unit 31A, an image processing unit 31B, a recording controller 31C, an output controller 31D, and the like. The functions of the respective units are realized by the processor 31 executing a predetermined program.

The data acquisition unit 31A acquires detection data of X-rays from the scanner gantry 10. As described above, the detection data is data in which the count value of the X-ray photon is assigned for each energy bin. The detection data includes information on a channel number and a column number of the detection element, a view number indicating a collected view, and a count value for each energy bin of the detected X-ray photon.

The image processing unit 31B generates a tomographic image by performing predetermined reconstruction processing on the detection data acquired by the data acquisition unit 31A. In addition, the image processing unit 31B generates spectral images such as a virtual monochromatic X-ray image, an effective atomic number image, and a material discrimination image in response to an instruction from the user. Each image is generated for each slice.

The recording controller 31C records the tomographic image (including the spectral image) generated by the image processing unit 31B, in the auxiliary memory 33. The image is recorded in an examination unit. That is, a plurality of tomographic images obtained in one examination are recorded in association with each other. In addition, the detection data of the generation source is recorded in association with each tomographic image.

The output controller 31D outputs the tomographic image (including the spectral image) generated by the image processing unit 31B, to the display device 35. In addition, the output controller 31D outputs the recorded tomographic image to the display device 35. The tomographic image is displayed in a predetermined format.

### (b) Analysis Function

The console 30 according to the present embodiment has a function of analyzing the shape and the size of a specific tissue (analysis function), as a function of supporting the interpretation.

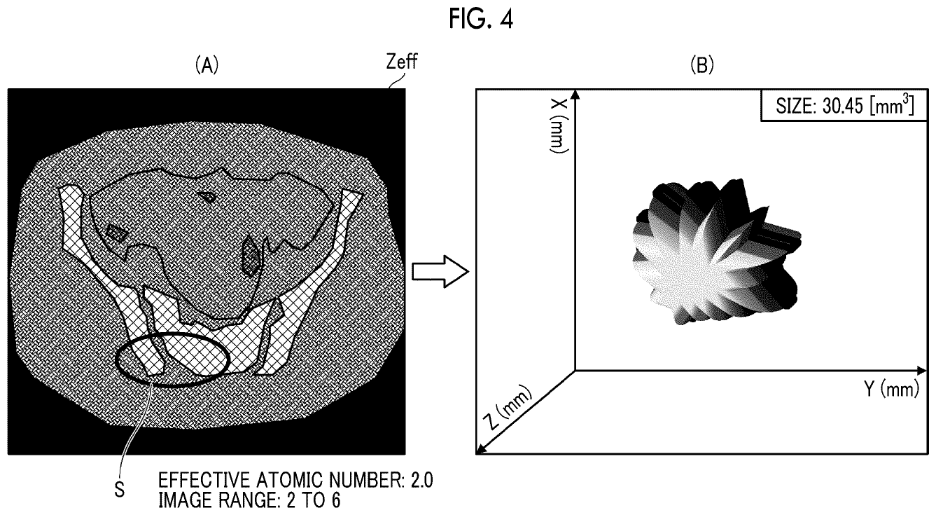

FIG. 4 is a conceptual diagram of the analysis function. In FIG. 4, (A) is a conceptual diagram of setting the analysis condition, and (B) is a diagram illustrating an example of the display of the analysis result.

The analysis of the shape and the size of the specific tissue is performed using the effective atomic number image.

As illustrated in (A) of FIG. 4, the user sets a region (analysis target region) S as the analysis target, on an effective atomic number image Zeff. The analysis target region S is synonymous with a region of interest (ROI).

In addition, the user sets the component of the tissue as the analysis target and the image range.

The component of the tissue as the analysis target is set by an effective atomic number. That is, the effective atomic number corresponding to the component of the tissue as the analysis target is set.

The image range is set by a slice number. The slice number is a serial number given to each tomographic image in an imaging (scanning) order.

The console 30 analyzes the effective atomic number image in the set image range, and extracts a region of a tissue having a set component, from the analysis target region of each image. The extraction is performed by extracting a pixel including the component having the set effective atomic number. The console 30 specifies a three-dimensional shape of the tissue and measures the volume thereof on the basis of the extraction result from each image. Then, the information on the specified shape and the measured volume is output as the analysis result. (B) of FIG. 4 illustrates an example of a case where a three-dimensional shape of the tissue is displayed in three dimensions as the analysis result.

In this manner, the tissue having the set component (effective atomic number) is extracted using the effective atomic number image, the shape and the size thereof are obtained, and the result is displayed. As a result, for example, the shape and size of each component of the stone or the like can be specified.

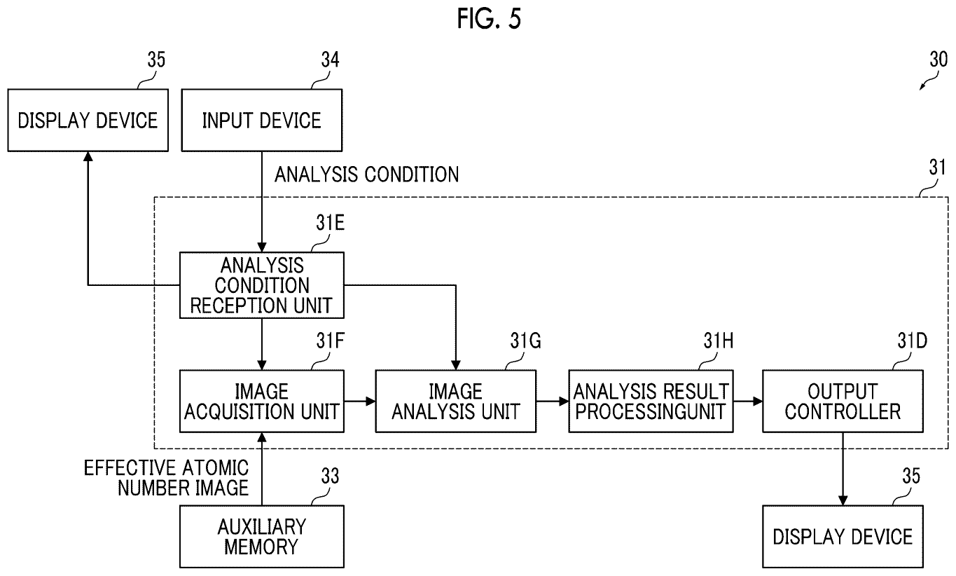

FIG. 5 is a block diagram of main functions of the console regarding the analysis function.

As illustrated in FIG. 5, regarding the analysis function, the console 30 has functions of an analysis condition reception unit 31E, an image acquisition unit 31F, an image analysis unit 31G, an analysis result processing unit 31H, the output controller 31D, and the like. The functions of the respective units are implemented by the processor 31 executing a predetermined program (image processing program).

### (a) Analysis Condition Reception Unit

The analysis condition reception unit 31E receives setting of analysis conditions from the user. Specifically, the analysis condition reception unit receives setting of the region as the analysis target (analysis target region), the component (effective atomic number) of the tissue as the analysis target, and the image range, from the user. A plurality of analysis target regions can be set. In a case where a plurality of analysis target regions are set, the analysis conditions (effective atomic number and image range) are set for each analysis target region. In the present embodiment, the analysis target region is an example of a first region.

The analysis condition reception unit 31E displays a predetermined setting screen on the display device 35 to receive the setting of the analysis conditions from the user.

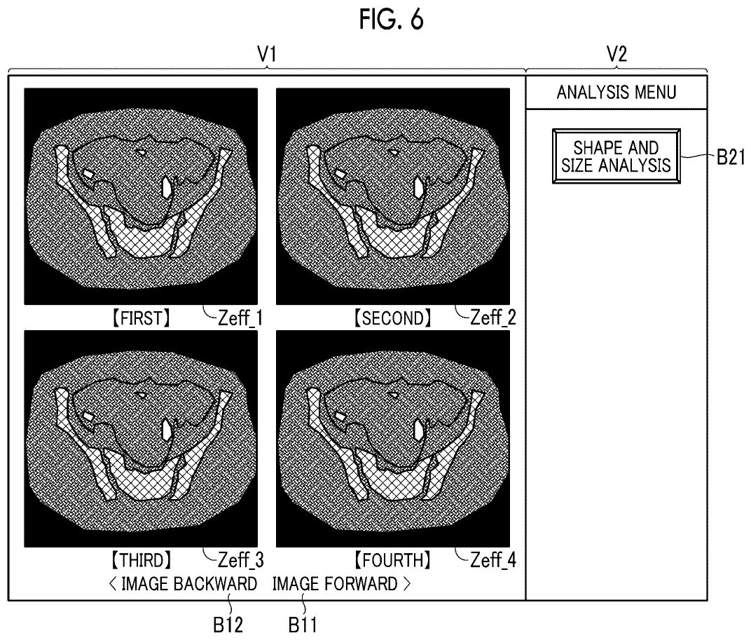

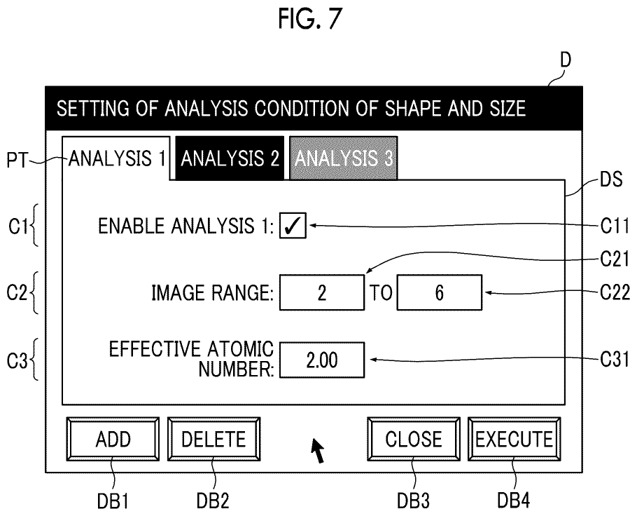

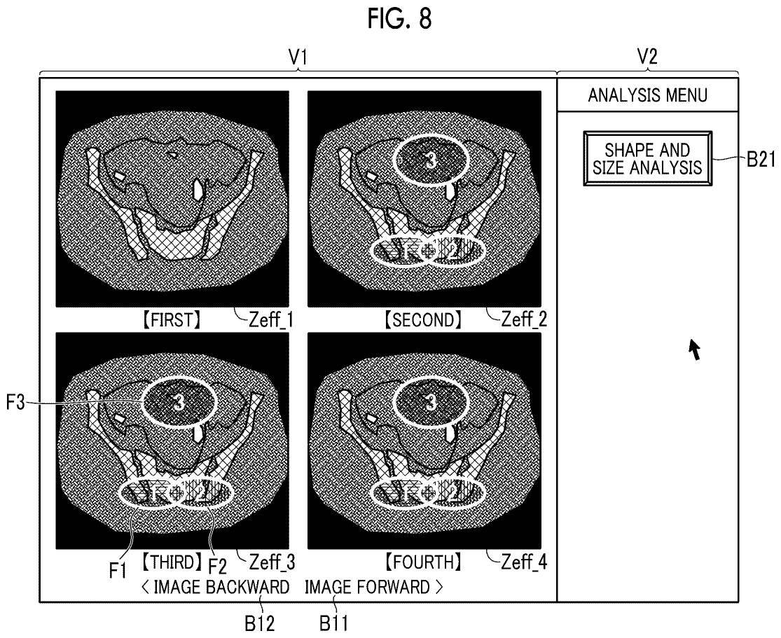

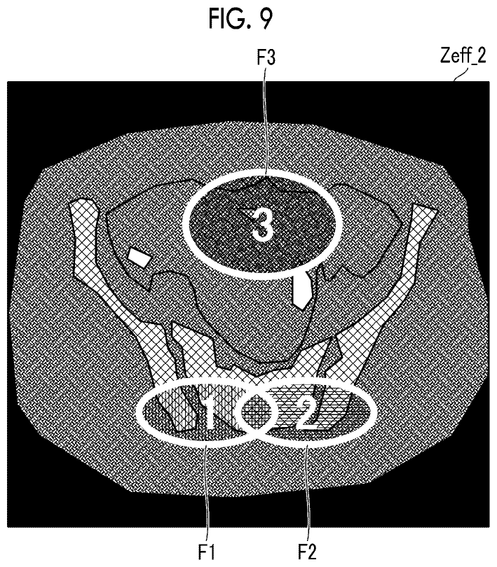

FIGS. 6 to 9 are diagrams illustrating an example of display of a screen in a case of performing the setting of the analysis conditions. FIG. 6 is a diagram illustrating an example of a display screen of the effective atomic number image. FIG. 7 is a diagram illustrating an example of a setting screen (analysis condition setting dialog) of the analysis condition. FIG. 8 is a diagram illustrating an example of a setting screen of the analysis target region. FIG. 9 is a diagram illustrating an example of the display of a frame (region setting frame) for setting the analysis target region.

In the PCCT device 1 according to the present embodiment, a function (analysis function) of analyzing the shape and size of a specific tissue is provided as an analysis menu for the effective atomic number image.

As illustrated in FIG. 6, an image display region V1 and a menu display region V2 are set on the display screen of the effective atomic number image.

In the image display region V1, effective atomic number images Zeff_1 to Zeff_4 are displayed. The example illustrated in FIG. 6 illustrates an example of a case where four effective atomic number images Zeff_1 to Zeff_4 are displayed at one time. The number of images to be displayed at one time is not limited to this, and for example, a configuration in which only one image is displayed may be adopted. An image forward button B11 and an image backward button B12 are displayed in the image display region V1. The image displayed in the image display region V1 is switched one by one by clicking the image forward button B11 or the image backward button B12. Note that a configuration in which a plurality of (for example, four) images are switched at once may be adopted. The images are displayed in an order of slice numbers, and are switched in the order of slice numbers.

A button for an item of the analysis that can be executed on the image being displayed in the image display region V1 is displayed in the menu display region V2. In the PCCT device 1 according to the present embodiment, since the analysis of the shape and size of the specific tissue is possible, a button (shape and size analysis button) B21 of the function is displayed in the menu display region V2. In a case of performing the analysis of the shape and size of the specific tissue, the user clicks the shape and size analysis button B21 displayed in the menu display region V2.

In a case where the shape and size analysis button B21 is clicked, a dialog (analysis condition setting dialog) D for setting the analysis condition is displayed in a pop-up manner on the screen.

As illustrated in FIG. 7, the analysis condition setting dialog D is provided with a sheet (analysis condition setting sheet) DS for setting the analysis conditions for each analysis target region. FIG. 7 illustrates an example of a case where analysis target regions are set at three locations. The switching of the sheet to be displayed is performed by a tab PT. FIG. 7 illustrates an example of a case where the analysis condition setting sheet DS of “analysis 1” is selected.

The analysis condition setting sheet DS includes a field C1 for enabling or disabling the analysis, a field C2 for setting the image range, and a field C3 for setting the effective atomic number.

A check box C11 is provided in the field C1 for enabling or disabling the analysis. By checking the check box C11, the analysis is enabled.

In the field C2 for setting the image range, a text box C21 for inputting a start point of the image range and a text box C22 for inputting an end point of the image range are provided. In the text box C21 for inputting the start point of the image range, the slice number of the effective atomic number image, which is the start of the image range, is input. In the text box C22 for inputting the end point of the image range, the slice number of the effective atomic number image, which is the end of the image range, is input. FIG. 7 illustrates an example of a case where the second to sixth effective atomic number images are set as the analysis targets. For example, in the text box C21 for inputting the start point of the image range, “1” is automatically input as a default numerical value. In addition, in the text box C22 for inputting the end point of the image range, for example, the slice number of the final effective atomic number image is automatically input as a default numerical value. That is, as the default setting, the effective atomic number images in the entire range are set as the analysis targets. The user narrows down the image range as the analysis target by changing the numerical value in the text box as necessary. Note that only one image can be designated in the image range. In a case where only one image is designated, the slice number of the image as the analysis target is input only in one text box C21, and the other text box C22 is left blank.

A text box C31 for inputting the effective atomic number is provided in the field C3 for setting the effective atomic number. FIG. 7 illustrates an example of a case where “2.00” is set as the effective atomic number as the analysis target.

As described above, a plurality of analysis target regions can be set. The analysis condition setting dialog D includes a button (addition button) DB1 for adding the analysis condition setting sheet DS and a button (deletion button) DB2 for deleting the analysis condition setting sheet DS. The number of default analysis condition setting sheets DS displayed is one. Each time the addition button DB1 is clicked, the analysis condition setting sheet DS is added. In addition, in a case where the deletion button DB2 is clicked, the analysis condition setting sheet DS being displayed is deleted.

A name (analysis name) for the analysis is assigned as a sheet name to the analysis condition setting sheet DS. The analysis names are automatically generated as “analysis 1”, “analysis 2”, . . . , and “analysis N” (N=1, 2, . . . ) in the order of sheet creation. The analysis name (sheet name) is displayed on the tab PT of each analysis condition setting sheet DS.

In a case where the check box C11 in the field C1 for enabling or disabling the analysis is checked and the analysis is enabled, as illustrated in FIG. 8, frames (region setting frames) F1 to F3 for setting the analysis target region are displayed on the effective atomic number images being displayed in the image display region V1. The region setting frames F1 to F3 are displayed on the images in the set image range. FIG. 8 illustrates an example of a case where the second to sixth effective atomic number images (effective atomic number images having slice numbers of 2 to 6) are set as the analysis targets for all the analysis target regions. In this case, the region setting frames F1 to F3 are displayed on the second to fourth effective atomic number images Zeff_2 to Zeff_4 (effective atomic number images having slice numbers of 2 to 4) among the effective atomic number images Zeff_1 to Zeff_4 (effective atomic number images having slice numbers of 1 to 4) being displayed in the image display region V1.

FIG. 9 is a diagram illustrating an example of the display of the region setting frame.

For example, as the initial display, the region setting frames F1 to F3 are displayed in a predetermined size circle and are displayed at the center of the image. The user sets the analysis target region at any position by adjusting the position, the size, and the shape (the aspect ratio of an ellipse) of the region setting frames F1 to F3 displayed in a circle. The adjustment of the position, the size, and the shape is performed by, for example, a mouse operation. The adjustment of the position, the size, and the shape can be performed on any image as long as the region setting frames F1 to F3 are displayed in the image. For example, in the example illustrated in FIG. 8, the region setting frames F1 to F3 can be adjusted in any of the second to fourth effective atomic number images Zeff_2 to Zeff_4. The adjustment performed on one image is also reflected on other images.

In each of the region setting frames F1 to F3, a number corresponding to the analysis name of the analysis condition setting sheet DS is displayed in the frame. In FIG. 9, the first region setting frame F1 is a frame corresponding to the analysis condition setting sheet of “analysis 1”. In addition, the second region setting frame F2 is a frame corresponding to the analysis condition setting sheet of “analysis 2”. In addition, the third region setting frame F3 is a frame corresponding to the analysis condition setting sheet of “analysis 3”.

In a case where a plurality of analysis target regions are set, it is preferable that the respective region setting frames are displayed in different colors. In addition, in a case where the respective region setting frames are displayed in different colors, it is preferable that the color (background color) of the corresponding analysis condition setting sheet DS (or the tab thereof) is displayed in the same color. For example, in a case where the first region setting frame F1 is displayed in red, the second region setting frame F2 is displayed in blue, and the third region setting frame F3 is displayed in yellow, the color of the analysis condition setting sheet of “analysis 1” is displayed in red, the color of the analysis condition setting sheet of “analysis 2” is displayed in blue, and the color of the analysis condition setting sheet of “analysis 3” is displayed in yellow. As a result, it is possible to clarify a correspondence relationship between the region setting frame and the analysis target sheet, and improve the convenience in a case of setting the analysis condition.

As illustrated in FIG. 7, the analysis condition setting dialog D includes a button (close button) DB3 for giving an instruction to close the dialog and a button (execution button) DB4 for giving an instruction to execute the analysis. The analysis condition setting dialog D is closed (erased from the screen) by clicking the close button DB3. The closed analysis condition setting dialog D is displayed again on the screen by clicking the shape and size analysis button B21 again. The execution of the analysis is instructed by clicking the execution button DB4.

### (b) Image Acquisition Unit

The image acquisition unit 31F acquires an image as the analysis target. The image acquisition unit 31F acquires, as the image as the analysis target, the effective atomic number image of the image range received by the analysis condition reception unit 31E, from the auxiliary memory 33.

### (c) Image Analysis Unit

The image analysis unit 31G analyzes the image designated as the analysis target, and extracts a region (corresponding tissue region) including the component of the effective atomic number designated as the analysis target, from the region (analysis target region) designated as the analysis target. Since the image range as the analysis target and the effective atomic number as the analysis target are set for each analysis target region, the corresponding tissue region is extracted for each analysis target region.

The extraction of the corresponding tissue region is performed by extracting a pixel including the component of the designated effective atomic number, from a correction target region of each image. For example, in a case where the effective atomic number 2.00 is designated as the analysis target for a first analysis target region, a pixel including the component of the effective atomic number 2.00 is extracted, and the corresponding tissue region is extracted. In the present embodiment, the corresponding tissue region is an example of a second region.

### (d) Analysis Result Processing Unit

The analysis result processing unit 31H specifies the shape of the region (corresponding tissue region) including the component of the effective atomic number designated as the analysis target and measures the size thereof, on the basis of the analysis result by the image analysis unit 31G. In a case where a plurality of images are designated as the analysis targets (in a case where an image range including a plurality of images is designated), the analysis result processing unit 31H specifies the shape of the corresponding tissue region as a three-dimensional shape and generates a three-dimensional image. The three-dimensional image is generated on the basis of information on the pixel extracted as the corresponding tissue region.

The size is calculated on the basis of the image resolution (pixel/mm). In a case where the shape of the corresponding tissue region is specified as a three-dimensional shape, the analysis result processing unit 31H calculates the volume of the region. In a case where the shape of the corresponding tissue region is specified by a plane (in a case of only one image as the analysis target), the analysis result processing unit 31H calculates the area of the region.

### (e) Output Controller

The output controller 31D outputs the analysis result to the display device 35. The output controller 31D outputs the information on the shape and the information on the size of the region extracted as the corresponding tissue region, in a predetermined format.

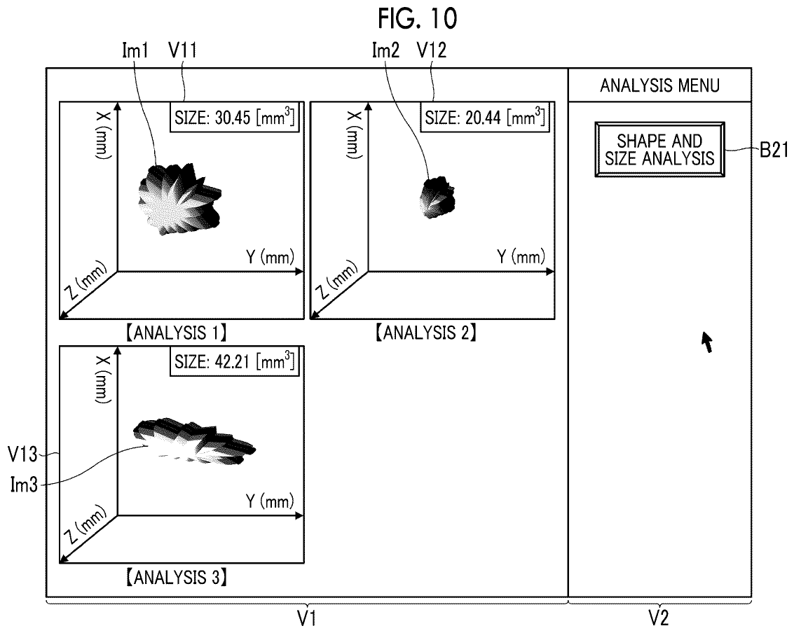

FIG. 10 is a diagram illustrating an example of the screen display of the analysis result.

As illustrated in FIG. 10, the analysis result is displayed in the image display region V1. In the image display region V1, analysis result display frames V11 to V13 are displayed according to the number of set analysis target regions. FIG. 10 illustrates an example of a case where analysis target regions are set at three locations. In the first analysis result display frame V11, the analysis result in the first analysis target region (analysis 1) is displayed. In the second analysis result display frame V12, the analysis result in the second analysis target region (analysis 2) is displayed. In the third analysis result display frame V13, the analysis result in the third analysis target region (analysis 3) is displayed.

In the frame, three-dimensional coordinates are displayed, and three-dimensional images Im1 to Im3 of the corresponding tissue region extracted from the corresponding analysis target region are displayed. In addition, the information on the measured size is displayed. The three-dimensional images Im1 to Im3 displayed in respective frames are individually enlarged, reduced, rotated, and the like in response to the instruction from the user.

### Processing Operation of Analysis

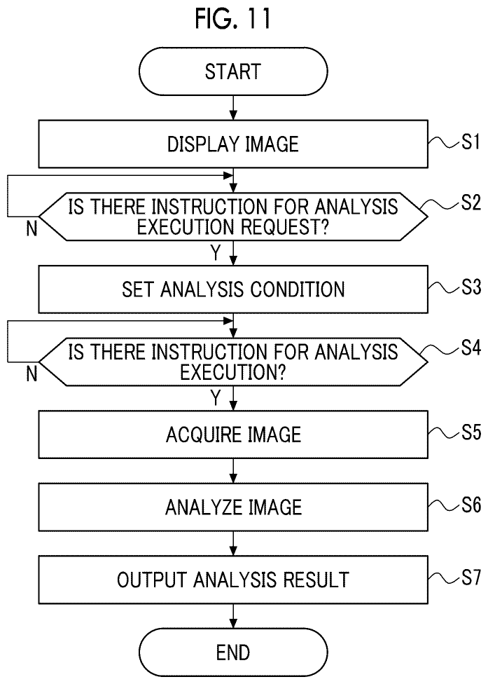

FIG. 11 is a flowchart illustrating an operation procedure in a case of performing processing of analyzing the shape and size of the specific tissue.

First, the effective atomic number image is displayed (step S1). As illustrated in FIG. 6, the effective atomic number image is displayed in the image display region V1.

Next, it is determined whether or not there is an execution request of an analysis (step S2). The execution request of the analysis is made by clicking the shape and size analysis button B21. The processor 31 determines whether or not there is an execution request of the analysis by determining whether or not the shape and size analysis button B21 is clicked.

In a case where the shape and size analysis button B21 is clicked and the execution request of the analysis is received, the analysis condition is set (step S3). The processor 31 displays the analysis condition setting dialog D on the screen, and receives the setting of the analysis condition (refer to FIG. 7). The user performs the designation of the image range, the designation of the effective atomic number, and the setting of enabling or disabling the analysis, in the analysis condition setting dialog D displayed on the screen. In a case where the analysis target regions are set at a plurality of locations, the analysis condition setting sheet DS is added and displayed. The region setting frame is displayed on the image of the corresponding image range with respect to the sheet in which the analysis is enabled (the analysis condition setting sheet in which the check box C11 is checked) (refer to FIGS. 8 and 9). The user sets the analysis target region by adjusting the position, size, and shape (aspect ratio) of the region setting frame displayed on the effective atomic number image.

After the setting of the analysis condition is completed, the user gives an instruction to execute the analysis by clicking the execution button DB4 displayed in the analysis condition setting dialog D. The processor 31 determines whether or not there is an instruction for the analysis execution by determining whether or not the execution button DB4 is clicked (step S4).

In a case where the instruction to execute the analysis is given, the effective atomic number image as the analysis target is acquired (step S5). The effective atomic number image as the analysis target is an effective atomic number image of the image range designated by the user.

The image analysis is performed on the acquired effective atomic number image under the designated condition (step S6). That is, the region (corresponding tissue region) including the component having the designated effective atomic number is extracted from the designated region (analysis target region). Then, the shape of the corresponding tissue region is specified and the size thereof is measured on the basis of the extraction result.

After the analysis is completed, the analysis result is output to the display device 35 (step S7). As illustrated in FIG. 10, the analysis result is displayed for each analysis target region, and a three-dimensional image of the extracted corresponding tissue region is displayed. In addition, the information on the measured size is displayed.

As described above, according to the present embodiment, the shape and size of the tissue having the component having the designated effective atomic number can be specified and displayed on the screen. As a result, for example, regarding the stone or the like, the shape and size thereof can be obtained for each component. For example, as the main components of the ureteral stone, struvite, uric acid, cystine, calcium oxalate, calcium phosphate, and the like are known. The effective atomic number of struvite is known to be 9.72, the effective atomic number of uric acid is known to be 6.92, the effective atomic number of cystine is known to be 11.07, the effective atomic number of calcium oxalate is known to be 13.52, and the effective atomic number of calcium phosphate is known to be 15.95. Therefore, by designating the effective atomic number of the component of interest, it is possible to discriminate the presence or absence of the component, and in a case where the tissue of the component is present, it is possible to know the shape and size of the component. For example, in a case where a uric acid stone is suspected, by specifying the effective atomic number of 9.62 and executing the analysis, it is possible to determine the presence or absence of the stone, and it is possible to ascertain the shape and size in a case where the stone is present. As described above, according to the present embodiment, for the stone of which the treatment method and the prevention method vary depending on the components, the shape and size can be identified for each component. As a result, the accuracy of the treatment and the prevention diagnosis can be improved.

### Modification Example

### Display Image

### (a) Type of Display Image

In the above-described embodiment, the effective atomic number image is output to the display device 35, and the analysis target region is set on the effective atomic number image displayed on the screen. However, the image output to the display device 35 is not limited thereto. For example, a normal CT image may be output to the display device 35, and the analysis target region may be set on the normal CT image displayed on the screen.

### (b) Display of Three-Dimensional Image

In the above-described embodiment, the two-dimensional tomographic image is displayed on the screen and the setting of the analysis target region is received. However, a three-dimensional image may be generated from the two-dimensional tomographic image obtained by imaging (scanning), the generated three-dimensional image may be displayed on the screen, and the setting of the analysis target region may be received. Since the generation of the three-dimensional image is a known technology, the details thereof will not be described.

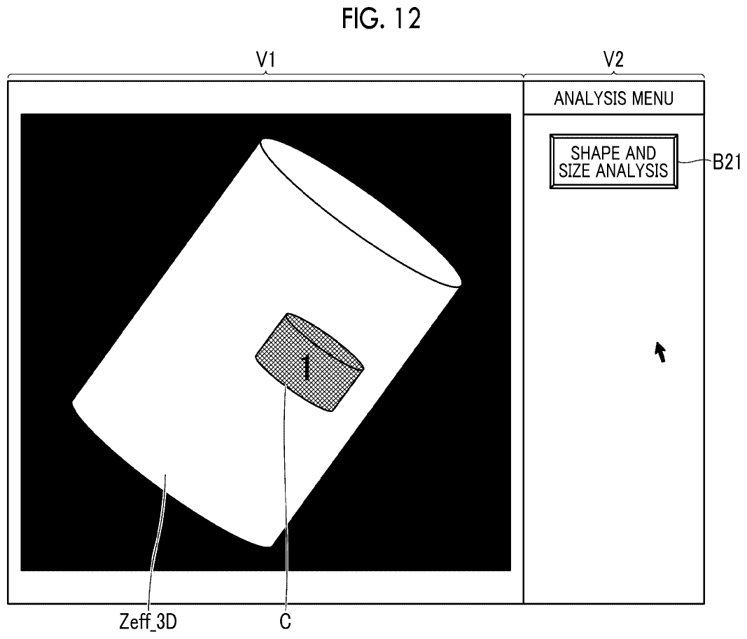

FIG. 12 is a diagram illustrating an example of the screen display in a case of setting the analysis target region on the three-dimensional image.

As illustrated in FIG. 12, a three-dimensional image Zeff_3D generated from the effective atomic number image is displayed in the image display region V1, and the setting of the analysis target region is received.

The analysis target region is set, for example, by displaying a cylinder C along the body axis direction and adjusting the position (position in a plane orthogonal to the body axis direction), the size (size of the cross section), and the shape (shape of the cross section) of the cylinder C. The region surrounded by the cylinder C is set as the analysis target region. Note that the position and the length of the cylinder C in the body axis direction are set in the image range. The image range may be set in the analysis condition setting dialog D or may be set on the screen. In a case of setting the image range on the screen, the setting is performed by adjusting the position and the length (height) of the cylinder C in the body axis direction.

### Setting of Analysis Condition

### (a) Setting Method of Effective Atomic Number

In the above-described embodiment, as a method of setting the effective atomic number as the analysis target, the text box C31 is provided in the field C3 for setting the effective atomic number of the analysis condition setting dialog D, and the user directly inputs the effective atomic number as the analysis target to the text box C31. The method of setting the effective atomic number as the analysis target is not limited thereto. For example, a plurality of effective atomic numbers may be registered in advance, and may be called and set as appropriate (so-called preset). Specifically, a plurality of effective atomic numbers are registered in advance as selection candidates, and are presented to the user at the time of setting, and one effective atomic number is selected. In a case of selection, it is preferable to display the information on the corresponding component in association with the effective atomic number. For example, the effective atomic number and the component corresponding to the effective atomic number are displayed in parallel. In addition, the registration is preferably performed by the classification into a plurality of categories. For example, even for the same type of stone, the primary components differ depending on the type (ureteral stone, kidney stone, bladder stone, gallstone, or the like), and therefore, it is preferable to perform the classification and the registration for each type. In addition, in a case where the registration is performed by classification, it is preferable that the user can select the classification. Furthermore, it is preferable that the registration can be optionally added, changed, deleted, and the like by the user.

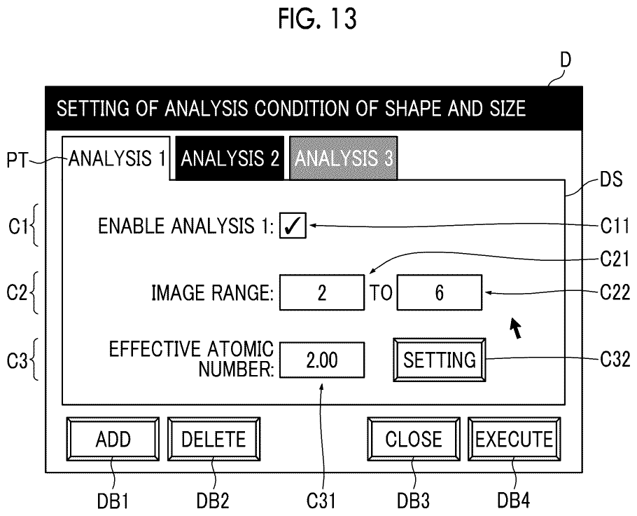

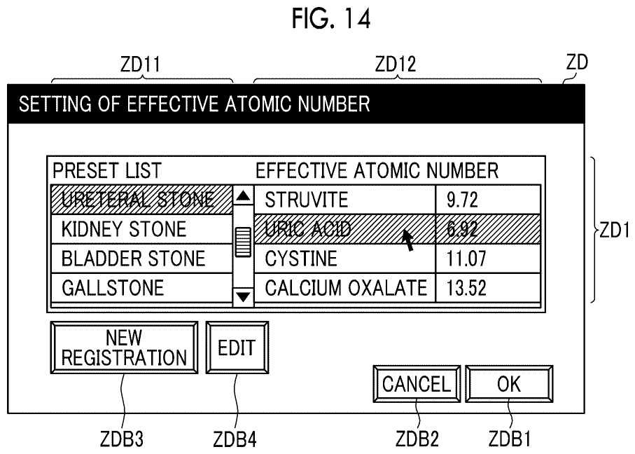

FIG. 13 is a diagram illustrating an example of the setting screen (analysis condition setting dialog) of the analysis condition in a case of setting the effective atomic number using the preset. In addition, FIG. 14 is a diagram illustrating an example of the screen (effective atomic number setting dialog) for setting the effective atomic number using the preset.

As illustrated in FIG. 13, in a case where the effective atomic number is set using the preset, a setting button C32 is provided in the analysis condition setting dialog D. The setting button C32 is a button for calling an effective atomic number setting screen. The setting button C32 is provided in the field C3 for setting the effective atomic number. In a case where the setting button C32 is clicked, an effective atomic number setting dialog ZD is displayed in a pop-up manner.

As illustrated in FIG. 14, the effective atomic number setting dialog ZD is provided with a field (effective atomic number selection field) ZD1 for selecting the effective atomic number.

The effective atomic number selection field ZD1 is provided with a field ZD11 for selecting a main classification and a field ZD12 for selecting the effective atomic number. In the field ZD11 for selecting the main classification, selectable main classifications are displayed in a list. In the field ZD12 for selecting the effective atomic number, the selectable effective atomic numbers in the main classification selected in the field ZD11 for selecting the main category are displayed in a list. The effective atomic number is displayed together with the information on the corresponding component. More specifically, the component and the effective atomic number thereof are displayed in parallel. The main classification is performed according to, for example, a disease name. FIG. 14 illustrates an example of a case of performing the classification according to the type of stone. The effective atomic numbers that can be selected for each main classification are registered. For example, FIG. 14 illustrates an example of a case where “ureteral stone” is selected as the main classification. Then, an example of a case where, regarding the “ureteral stone” that is the main classification, “struvite: 9.72”, “uric acid: 6.92”, “cystine: 11.07”, “calcium oxalate: 13.52”, and “calcium phosphate: 15.95” are registered as selectable effective atomic numbers is illustrated. After the main classification is selected, the user selects the effective atomic number as the analysis target from the effective atomic numbers displayed in a list in the field ZD12 for selecting the effective atomic number. The background color of the selected main classification and effective atomic number is changed (for example, inverted).

The effective atomic number setting dialog ZD includes an OK button ZDB1, a cancel button ZDB2, a new registration button ZDB3, and an edit button ZDB4.

The OK button ZDB1 is a button for instructing the reflection (application) of the setting. In a case where the OK button ZDB1 is clicked, the selected effective atomic number is reflected in the setting of the analysis condition. Specifically, the selected effective atomic number is automatically input to the text box C31 of the field C3 for setting the effective atomic number of the analysis condition setting dialog D. In a case where the OK button ZDB1 is clicked, the effective atomic number setting dialog ZD is erased from the screen. In a case of re-selecting, the setting button C32 is clicked again in the analysis condition setting dialog D.

The cancel button ZDB2 is a button for instructing the canceling of the setting operation of the effective atomic number by the preset. In a case where the cancel button ZDB2 is clicked, the processing is interrupted, and the effective atomic number setting dialog ZD is erased from the screen.

The new registration button ZDB3 is a button for instructing the execution of new registration. In a case where the new registration button ZDB3 is clicked, a predetermined registration screen is displayed, and the registration of the effective atomic number can be performed. The registration processing includes processing of adding a main classification and processing of newly adding an effective atomic number to the main classification of which the registration has already been created. In a case of adding a main classification, a name (classification name) of a newly added main classification is assigned, and an effective atomic number that can be selected in the main classification and a name of a component corresponding to the effective atomic number are registered. In a case of adding a new effective atomic number to the main classification of which the registration has already been created, the main classification is selected, and an effective atomic number to be added and a name of a component corresponding to the effective atomic number are registered.

The edit button ZDB4 is a button for instructing the editing of the registered effective atomic number. In a case where the edit button ZDB4 is clicked, a predetermined edit screen is displayed, and the editing of the effective atomic number can be performed. The editing processing includes processing of changing the name of the main classification, processing of deleting the main classification, processing of deleting the registered effective atomic number, processing of correcting the effective atomic number of which the registration has already been created, processing of correcting the name of the component associated with the registered effective atomic number, and the like.

By using the preset function in this manner, the setting operation of the analysis condition can be easily performed, and the convenience can be improved.

### (b) Setting of Effective Atomic Number Having Width

In the above-described embodiment, the effective atomic number as the analysis target is designated with pinpoint accuracy, but may be set to have a width. For example, for the stone, the criterion for determining the value range of the effective atomic number considered as the stone may vary depending on the facility. By providing a width to the value of the effective atomic number that can be set, the range as the analysis target can be widened.

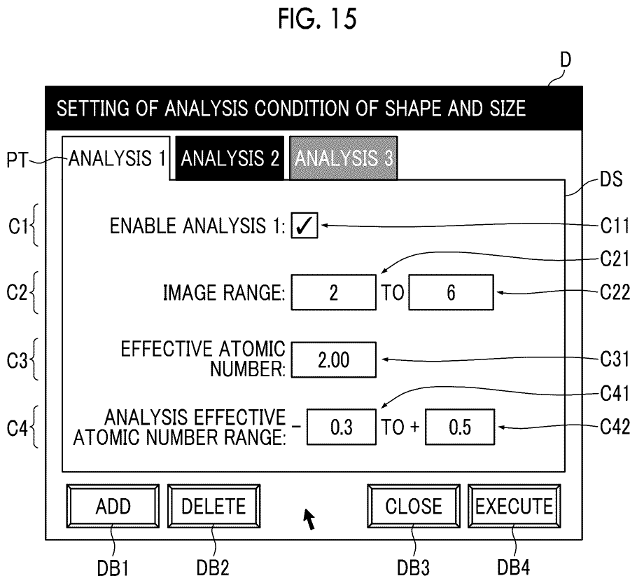

FIG. 15 is a diagram illustrating an example of the setting screen (analysis condition setting dialog D) for the analysis condition in a case of setting the effective atomic number having a width.

As illustrated in FIG. 15, in the analysis condition setting dialog D of the present example, a field C4 for setting a range of the effective atomic number is provided in a sheet (analysis condition setting sheet DS) for setting the analysis condition.

In this example, a configuration is adopted in which the range is designated by designating the range on the negative side and the range on the positive side with the effective atomic number set in the field C3 for setting the effective atomic number as a reference. Therefore, in the analysis condition setting dialog D of the present example, a text box C41 for inputting a range on the negative side and a text box C42 for inputting a range on the positive side are provided in the field C4 for setting the range of the effective atomic number.

In the text box C41 for inputting the range on the negative side, a numerical value of the range set on the negative side is input with the value of the effective atomic number set in the field C3 for setting the effective atomic number as a reference.

In the text box C42 for inputting the range on the positive side, a numerical value of the range set on the positive side is input with the effective atomic number set in the field C3 for setting the effective atomic number as a reference.

In the example illustrated in FIG. 15, an example of a case where “2.00” is designated as the reference effective atomic number, “0.3” (−0.3) is designated as the range on the negative side, and “0.5” (+0.5) is designated as the range on the positive side is illustrated. That is, an example of a case of performing the analysis in a range of −0.3 to +0.5 with 2.00 as a reference is illustrated. In this case, a range of 1.70 to 2.50 is set as the effective atomic number of the analysis target. Therefore, in a case where the execution of the analysis is instructed, the analysis is executed with the effective atomic number in a range of 1.70 to 2.50. That is, pixels including the component having the effective atomic number in a range of 1.70 to 2.50 are extracted, and the shape and the size are analyzed.

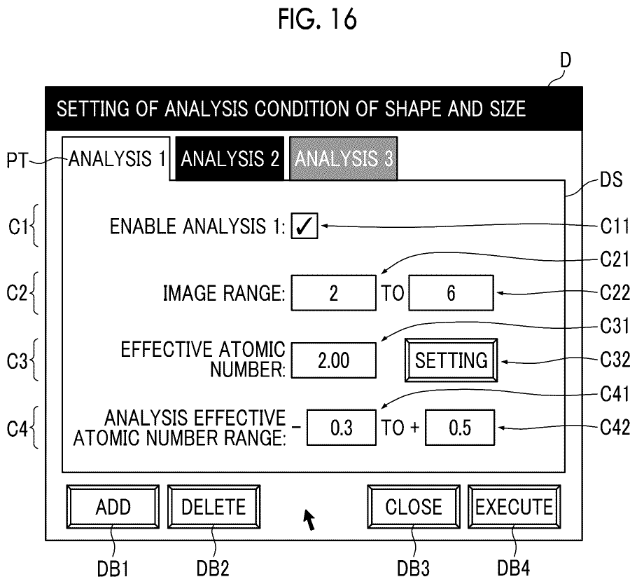

FIG. 16 is a diagram illustrating another example of the setting screen of the analysis condition in a case of setting the effective atomic number having a width.

FIG. 16 illustrates an example of a case of setting the reference effective atomic number using a preset. The setting of the effective atomic number by the preset is as described above.

In this example, with respect to the effective atomic number designated by the preset, the range of the effective atomic number as the analysis target is designated by designating the range on the negative side and the range on the positive side in the field C4 for setting the range of the effective atomic number.

In addition, a configuration may be adopted in which the range of the effective atomic number as the analysis target is directly input and set. In this case, for example, a field for setting the range of the effective atomic number is provided in the analysis condition setting sheet DS, and a text box for inputting the start point and the end point of the range is provided in the field.

### Result Output

In the above-described embodiment, the shape and the size of the tissue extracted by the analysis processing are output as the analysis result, but only one of the analysis results may be output. For example, only the analysis result of the shape may be output. In this case, the measurement of the size can be omitted.

In addition, in the above-described embodiment, the three-dimensional shape of the extracted tissue is specified as the analysis result of the shape, and the three-dimensional image thereof is generated and output, but the analysis result of the shape may be output in a format of a two-dimensional image. In this case, for example, the two-dimensional image of the corresponding tissue region extracted from each image within the image range designated as the analysis target is individually displayed.

In addition, in the above-described embodiment, the analysis result of the shape is independently displayed, but the analysis result of the shape may be displayed in a superimposed manner on the three-dimensional image (three-dimensional image generated from two-dimensional tomographic image obtained by imaging) as the analysis source.

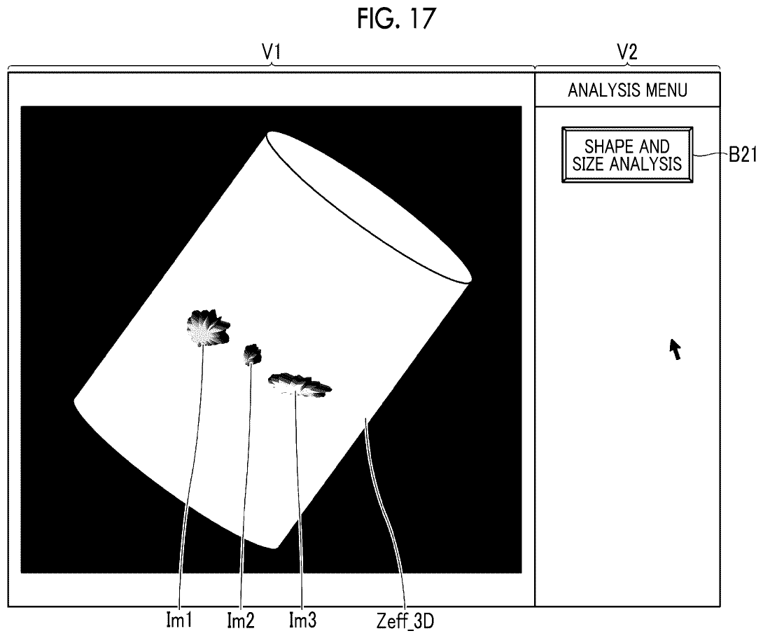

FIG. 17 is a diagram illustrating an example of a case of outputting the analysis result in a superimposed manner on the three-dimensional image as the analysis source.

As illustrated in FIG. 17, the three-dimensional image Zeff_3D as the analysis source is displayed in the image display region V1, and the three-dimensional images Im1 to Im3 of the corresponding tissue regions extracted from each analysis target region are displayed in a superimposed manner on the three-dimensional image Zeff_3D. The three-dimensional images Im1 to Im3 of the corresponding tissue regions are displayed on the three-dimensional image Zeff_3D as the analysis source with the positions and the sizes thereof aligned.

By outputting the analysis result in a superimposed manner on the three-dimensional image as the analysis source in this manner, it is possible to easily ascertain the position and the size thereof.

Note that, in the above example, the case where the analysis result is output in a superimposed manner on the three-dimensional image as the analysis source has been described. However, for example, a three-dimensional image of a specific organ, a specific structure, or the like may be newly generated from the two-dimensional tomographic image obtained by imaging, and the analysis result may be displayed in a superimposed manner on the three-dimensional image. In addition, for example, a three-dimensional image of the region designated as the analysis target region may be generated, and the analysis result may be displayed in a superimposed manner on the three-dimensional image. It is preferable to adopt a plurality of display forms so that the user can select any display form. For example, it is preferable that the individual display of the results and the display in a superimposed manner on the three-dimensional image can be freely switched.

In addition, in the above example, the case where only the analysis result of the shape is output has been described as an example, but in a case where the size is measured, it is preferable to also display information on the measurement result.

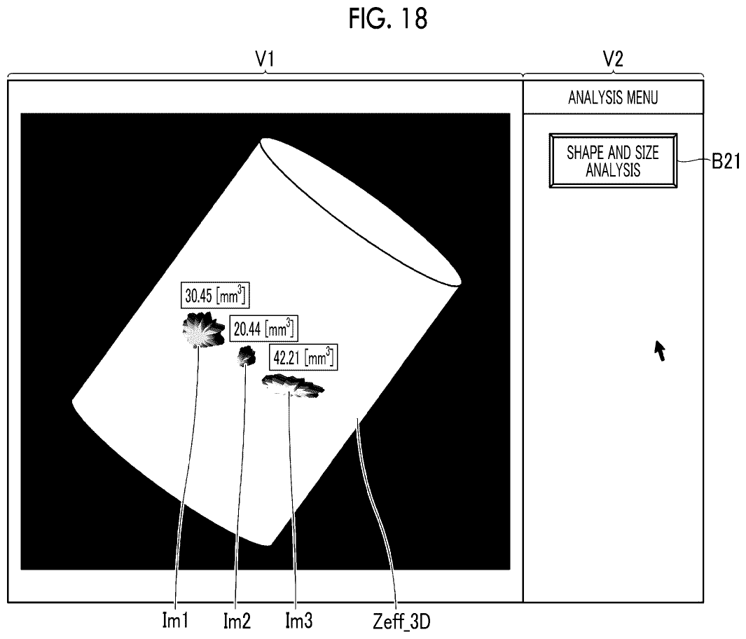

FIG. 18 is a diagram illustrating an example of a case of also displaying the measurement result of the size in a case where the analysis result is output in a superimposed manner on the three-dimensional image as the analysis source.

As illustrated in FIG. 18, the measurement result of the size is displayed in the vicinity of the three-dimensional images Im1 to Im3 of the corresponding tissue regions. In addition, a region for displaying the measurement result of the size may be provided on the screen, and the measurement result of the size may be displayed in the region.

In this manner, by displaying the measurement result of the size in addition to the analysis result of the shape, it is possible to easily check the shape and the size of the target tissue.

### X-Ray CT Device

In the above-described embodiments, a case where the present invention is applied to the PCCT device has been described as an example, but the application of the present invention is not limited to this. The present invention can be applied to an X-ray CT apparatus as long as the X-ray CT device can measure the effective atomic number (X-ray CT apparatus can reconstruct the effective atomic number image from the detection data obtained by imaging). For example, in a case of an X-ray CT device (for example, a spectral CT device) having a function of reconstructing a CT image in which the material discrimination is possible by detecting X-rays transmitted through a subject at two or more energy levels, the effective atomic number image can be acquired, and therefore, the present invention can be applied.

### Others

In the above-described embodiment, the console has the function of the image processing apparatus, but the image processing apparatus may be configured as an independent apparatus separate from the console.

The processing unit that provides the function of the image processing apparatus can be configured by various processors. The various processors include, in addition to a CPU and a graphic processing unit (GPU) as a general-purpose processor, a programmable logic device (PLD) which is a processor of which the circuit configuration can be changed after manufacturing, such as a field programmable gate array (FPGA), and a dedicated circuitry which is a processor having a circuit configuration specifically designed to execute specific processing, such as an application specific integrated circuit (ASIC). One processing unit may be configured of one of various processors or may be configured of two or more processors of the same type or different types. For example, one processing unit may be configured by a combination of a plurality of FPGAs or a combination of a CPU and an FPGA. In addition, a plurality of processing units may be configured of one processor. As an example of configuring a plurality of processing units with one processor, first, there is a form in which, as typified by computers used for a client, a server, or the like, one processor is configured by combining one or more CPUs and software, and the processor functions as a plurality of processing units. Second, as typified by a system on chip (SoC) or the like, a processor that realizes the functions of the entire system including the plurality of processing units by using one integrated circuit (IC) chip is used. As described above, the various processing units are configured using one or more of the various processors as a hardware structure.

### EXPLANATION OF REFERENCES

1: PCCT device
10: scanner gantry
10A: opening portion
11: X-ray tube device
12: X-ray detection device
13: data acquisition system
14: rotation frame
20: examination table
21: top plate
30: console
31: processor
31A: data acquisition unit
31B: image processing unit
31C: recording controller
31D: output controller
31E: analysis condition reception unit
31F: image acquisition unit
31G: image analysis unit
31H: analysis result processing unit
32: main memory
33: auxiliary memory
34: input device
35: display device
36: input and output interface
B11: image forward button
B12: image backward button
B21: analysis button
C: cylinder
C1: field for enabling or disabling analysis
C11: check box
C2: field for setting image range
C21: text box
C22: text box
C3: field for setting effective atomic number
C31: text box
C32: setting button
C4: field for setting range of effective atomic number
C41: text box
C42: text box
D: analysis condition setting dialog
DB1: addition button
DB2: deletion button
DB3: button
DB4: execution button
DS: analysis condition setting sheet
F1: first region setting frame
F2: second region setting frame
F3: third region setting frame
Im1: three-dimensional image of corresponding tissue region
Im2: three-dimensional image of corresponding tissue region
Im3: three-dimensional image of corresponding tissue region
P: subject
PT: tab
S: analysis target region
V1: image display region
V11: first analysis result display frame
V12: second analysis result display frame
V13: third analysis result display frame
V2: menu display region
ZD: effective atomic number setting dialog
ZD1: effective atomic number selection field
ZD11: field for selecting main classification
ZD12: field for selecting effective atomic number
ZDB1: OK button
ZDB2: cancel button
ZDB3: new registration button
ZDB4: edit button
Zeff: effective atomic number image
Zeff_1: effective atomic number image
Zeff_2: effective atomic number image
Zeff_3D: three-dimensional image
S1 to S7: operation procedure in case of executing processing of analyzing shape and size of specific tissue

What is claimed is:

1. An image processing apparatus that processes an image obtained by an X-ray computed tomography device capable of measuring an effective atomic number, the image processing apparatus comprising:
a processor,
wherein the processor
displays at least one of a plurality of images obtained by the X-ray computed tomography device on a display unit,
receives setting of a first region on the image displayed on the display unit,
receives setting of an effective atomic number as an analysis target,
extracts a pixel including a component of the effective atomic number in the first region and extracts a second region including the component of the effective atomic number in the first region,
specifies a shape of the second region, and
displays the shape of the second region on the display unit.

2. The image processing apparatus according to claim 1,
wherein the processor
measures a size of the second region, and
displays the shape and the size of the second region on the display unit.

3. The image processing apparatus according to claim 1,
wherein the processor receives setting of a range of the effective atomic number as the analysis target.

4. The image processing apparatus according to claim 3,
wherein the processor receives setting of a value of the effective atomic number as a reference and a range based on the value, and receives the setting of the range of the effective atomic number as the analysis target.

5. The image processing apparatus according to claim 1,
wherein the processor
displays, as the effective atomic number corresponding to a specific component, the effective atomic number registered in advance on the display unit, and
receives selection from the effective atomic number displayed on the display unit and receives the setting of the effective atomic number as the analysis target.

6. The image processing apparatus according to claim 5,
wherein the processor displays information on the component corresponding to the effective atomic number on the display unit in association with the effective atomic number.

7. The image processing apparatus according to claim 5,
wherein the processor receives registration of the effective atomic number to be displayed on the display unit.

8. The image processing apparatus according to claim 1,
wherein the processor
receives setting of a plurality of first regions,
receives setting of the effective atomic number as the analysis target for each first region,
extracts the second region for each first region,
specifies the shape of the second region for each first region, and
displays the shape of the second region on the display unit for each first region.

9. The image processing apparatus according to claim 8,
wherein the processor
measures a size of the second region for each first region, and
displays the shape and the size of the second region on the display unit for each first region.

10. The image processing apparatus according to claim 1,
wherein the processor
receives setting of a range of the image as the analysis target from among the plurality of images obtained by the X-ray computed tomography device,
individually extracts the second region from the image in the set range,
specifies a three-dimensional shape of the second region, and
displays the three-dimensional shape of the second region on the display unit.

11. The image processing apparatus according to claim 10,
wherein the processor
measures a volume of the second region and measures a size of the second region, and
displays the three-dimensional shape and the volume of the second region on the display unit.

12. The image processing apparatus according to claim 10,
wherein the processor
displays a three-dimensional image generated from the plurality of images obtained by the X-ray computed tomography device on the display unit, and
receives setting of the first region on the three-dimensional image displayed on the display unit.

13. The image processing apparatus according to claim 1,
wherein the processor displays a three-dimensional shape of the second region on a three-dimensional image generated from the plurality of images obtained by the X-ray computed tomography device.

14. The image processing apparatus according to claim 1,
wherein the X-ray computed tomography device is an X-ray computed tomography device capable of photon counting computed tomography.

15. The image processing apparatus according to claim 14,
wherein the image obtained by the X-ray computed tomography device is an effective atomic number image.

16. An image processing method of processing an image obtained by an X-ray computed tomography device capable of measuring an effective atomic number, the image processing method comprising:
displaying at least one of a plurality of images obtained by the X-ray computed tomography device on a display unit;
receiving setting of a first region on the image displayed on the display unit;
receiving setting of an effective atomic number as an analysis target;
extracting a pixel including a component of the effective atomic number in the first region and extracting a second region including the component of the effective atomic number in the first region;
specifying a shape of the second region; and
displaying the shape of the second region on the display unit.

17. A non-transitory, computer-readable tangible recording medium which records thereon, an image processing program for processing an image obtained by an X-ray computed tomography device capable of measuring an effective atomic number, the image processing program causing a computer to implement:
a function of displaying at least one of a plurality of images obtained by the X-ray computed tomography device on a display unit;
a function of receiving setting of a first region on the image displayed on the display unit;
a function of receiving setting of an effective atomic number as an analysis target;
a function of extracting a pixel including a component of the effective atomic number in the first region and extracting a second region including the component of the effective atomic number in the first region;
a function of specifying a shape of the second region; and
a function of displaying the shape of the second region on the display unit.
